(19) United States
(12) Patent Application Publication
(10) Pub. No.: US 2025/0291530 A1
(43) Pub. Date: Sep. 18, 2025

(54) INFORMATION MANAGEMENT SYSTEM, MANAGEMENT DEVICE, AND NON-TRANSITORY COMPUTER-READABLE STORAGE MEDIUM STORING PROGRAM

(71) Applicant: SEIKO EPSON CORPORATION, Tokyo (JP)

(72) Inventors: Yusuke NAKANO, MATSUMOTO-SHI (JP); Hiroyuki KURAMOTO, SHIOJIRI-SHI (JP)

(21) Appl. No.: 19/081,515

(22) Filed: Mar. 17, 2025

(30) Foreign Application Priority Data
Mar. 18, 2024 (JP) 2024-042102

(51) Int. Cl. G06F 3/12

(52) U.S. Cl. CPC G06F 3/1243; G06F 3/1206; G06F 3/1231

(57) ABSTRACT

An information management system includes: a management system that manages management information; a print system that causes a printing device to print the management information acquired from the management system; and a management device that acquires the management information from scan data of a printed object with the management information printed thereon, and stores the acquired management information in a database on an isolated network isolated from an external network, and the print system causes the printing device to generate the printed object with identification information in accordance with a predetermined order rule printed thereon, and the management device acquires the identification information from the scan data of the printed object and gives a notification of a storage omission of the management information in the database, based on the acquired identification information.

The present application is based on, and claims priority from JP Application Serial Number 2024-042102, filed Mar. 18, 2024, the disclosure of which is hereby incorporated by reference herein in its entirety.

### BACKGROUND

### 1. Technical Field

The present disclosure relates to an information management system, a management device, and a non-transitory computer-readable storage medium storing a program.

### 2. Related Art

According to the related art, a technique of managing information on a network is known. For example, JP-A-2022-063904 discloses a system in which a cloud server on a cloud network manages medical information such as an electronic medical record. JP-A-2022-063904 is an example of the related art.

Management information such as medical information described in JP-A-2022-063904 may be managed in a database on an isolated network. In this case, as a configuration to store the management information in the database, a configuration in which a scanner reads a printed object with the management information printed thereon and the management information acquired from the scan data is stored in the database, is conceivable. However, in this configuration, a storage omission of the management information in the database may occur due to a loss of a printed object, a multi-feed occurring in the scanner, or the like.

### SUMMARY

According to an aspect of the present disclosure, an information management system includes: a management system that manages management information; a print system that causes a printing device to print the management information acquired from the management system; and a management device that acquires the management information from scan data of a printed object with the management information printed thereon, and stores the acquired management information in a database on an isolated network isolated from an external network, the print system causing the printing device to generate the printed object with identification information in accordance with a predetermined order rule printed thereon, the management device acquiring the identification information from the scan data of the printed object and giving a notification of a storage omission of the management information in the database, based on the acquired identification information.

According to another aspect of the present disclosure, a management device is provided, the management device being connected to an isolated network isolated from an external network to which a management system that manages management information and a print system that causes a printing device to print the management information acquired from the management system are connected, the management device including a management device control unit that acquires identification information in accordance with a predetermined order rule from scan data of a printed object with the identification information printed thereon, and gives a notification of a storage omission of the management information in a database on the isolated network, based on the acquired identification information.

According to still another aspect of the present disclosure, a non-transitory computer-readable storage medium storing a program is provided, the program causing a processor of a management device connected to an isolated network isolated from an external network to which a management system that manages management information and a print system that causes a printing device to print the management information acquired from the management system are connected, to acquire identification information in accordance with a predetermined order rule from scan data of a printed object with the identification information printed thereon, and give a notification of a storage omission of the management information in a database on the isolated network, based on the acquired identification information.

According to still another aspect of the present disclosure, a server is provided, the server being connected to a management system that manages management information, via an external network, and causing a printing device to print the management information acquired from the management system, the server including a server control unit that causes the printing device to print a printed object on which identification information acquired when the management device connected to an isolated network isolated from the external network gives a notification of a storage omission of the management information in a database on the isolated network is printed, the identification information printed on the printed object is information in accordance with a predetermined order rule.

### BRIEF DESCRIPTION OF THE DRAWINGS

FIG. 1 shows a configuration of an information management system.

FIG. 2 shows a configuration of a medical questionnaire management server and a print server.

FIG. 3 shows an example of a print image.

FIG. 4 shows a configuration of a second in-hospital terminal.

FIG. 5 is a flowchart showing the operation of each unit of the information management system.

FIG. 6 is a flowchart showing the operation of each unit of the information management system.

FIG. 7 illustrates the estimation of a period of missing.

FIG. 8 shows an example of a notification screen.

FIG. 9 shows a configuration of a medical questionnaire management server and a print server.

FIG. 10 is a flowchart showing the operation of each unit of the information management system.

### DESCRIPTION OF EMBODIMENTS

### 1. First Embodiment

### 1-1. Configuration of Information Management System

First, a first embodiment will be described.

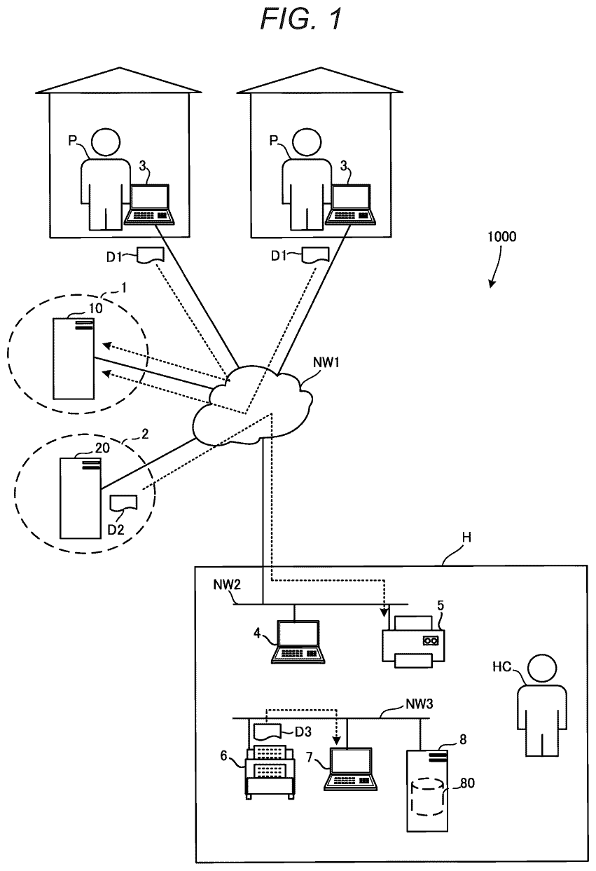

FIG. 1 shows a configuration of an information management system 1000.

The information management system 1000 according to the present embodiment is a system applied to, for example, a hospital H, and is a system that manages medical questionnaire answer information J1. The medical questionnaire answer information J1 is information of an answer by a patient P to a medical questionnaire. The medical questionnaire answer information J1 includes, for example, a medication history answered by the patient P, a medical history answered by the patient P, and a consultation history answered by the patient P.

The medical questionnaire answer information J1 is an example of “management information”. The hospital H is an example of a “facility”.

The hospital H in the present embodiment is an institution that provides medical care for the patient P, and may or may not be a facility that accommodates the patient P. That is, the hospital H in the present embodiment may be an institution that provides medical care for outpatients or may be an institution that provides medical care for outpatients and inpatients.

The information management system 1000 includes a medical questionnaire management system 1 connected to an external network NW1 which is a WAN (wide area network) such as the internet.

The medical questionnaire management system 1 manages the medical questionnaire answer information J1. The medical questionnaire management system 1 includes a medical questionnaire management server 10. The medical questionnaire management server 10 is a server device that manages the medical questionnaire answer information J1, and is a server device that processes information, using a print system 2, a patient terminal 3 used by the patient P, and a first in-hospital terminal 4 used in the hospital H, as clients. The medical questionnaire management server 10 connects to the external network NW1 and communicates with these devices. In each drawing, the medical questionnaire management server 10 is expressed by one block, but this does not necessarily mean that the medical questionnaire management server 10 is configured with a single device. For example, the medical questionnaire management server 10 may include a plurality of server devices having different processing contents.

The medical questionnaire management system 1 is an example of a “management system”.

The patient terminal 3 is a terminal device used by the patient P. Although FIG. 1 illustrates a case where the patient terminal 3 is a laptop PC, the patient terminal 3 may be a desktop PC, a tablet PC, or a smartphone. The patient terminal 3 is connected to the external network NW1 and communicates with the medical questionnaire management system 1. The patient terminal 3 displays, for example, an input screen for a medical questionnaire at the home or the like of the patient, and accepts an input of an answer to the medical questionnaire from the patient P. When the patient P finishes the answer to the medical questionnaire, the patient terminal 3 transmits medical questionnaire answer information data D1 representing the medical questionnaire answer information J1 to the medical questionnaire management system 1.

The information management system 1000 includes the print system 2 connected to the external network NW1.

The print system 2 is a system that causes a printing device 5 used in the hospital H to perform printing. The print system 2 includes a print server 20. The print server 20 is a server device that processes information, using the medical questionnaire management system 1 and the printing device 5 as clients. The print server 20 connects to the external network NW1 and communicates with these devices. In each drawing, the print server 20 is expressed by one block, but this does not necessarily mean that the print server 20 is configured with a single device. For example, the print server 20 may include a plurality of server devices having different processing contents.

The print server 20 is an example of a “server”.

The information management system 1000 includes the printing device 5.

The printing device 5 is a device that performs printing with a print head and thus generates a printed object. The printing device 5 is installed in the hospital H and connects to an external connection network NW2 built in the hospital H. The external connection network NW2 is a network connected to the external network NW1. Therefore, the printing device 5 can communicate with the first in-hospital terminal 4 connected to the external connection network NW2 and the print system 2 connected to the external network NW1. The printing device 5 in the present embodiment receives print data D2 from the print system 2 and performs printing based on the received print data D2. The print data D2 is data including image data of an image to be formed on a printed object and control commands conforming to the command system of the printing device 5.

The printing device 5 may be an inkjet-type printing device or an electronic transfer-type printing device. When the printing device 5 is an inkjet type, the format of the print head provided in the printing device 5 may be a serial type, a line type, or a lateral type. The printing device 5 may also have functions such as a scanning function and a facsimile function in addition to the printing function.

As shown in FIG. 1, in addition to the printing device 5, the first in-hospital terminal 4 is connected to the external connection network NW2.

The first in-hospital terminal 4 is a terminal device used by a healthcare worker HC. The healthcare worker HC includes a doctor, a nurse, a person engaged in clerical work related to the hospital H, and the like. Although FIG. 1 illustrates a case where the first in-hospital terminal 4 is a laptop PC, the first in-hospital terminal 4 may be a desktop PC or a tablet PC. The first in-hospital terminal 4 connects to the external connection network NW2 and communicates with the medical questionnaire management system 1 and the printing device 5.

The information management system 1000 includes a scanner 6.

The scanner 6 is a device that performs scanning. The scanner 6 is installed in the hospital H and connects to an isolated network NW3 built in the hospital H. The isolated network NW3 is a network isolated from the external network NW1. That is, the isolated network NW3 is not connected to the external network NW1. The scanner 6 transmits scan data D3 acquired by scanning to a second in-hospital terminal 7 connected to the isolated network NW3.

The second in-hospital terminal 7 is an example of a “management device”.

The information management system 1000 includes the second in-hospital terminal 7.

The second in-hospital terminal 7 is a terminal device installed in the hospital H and used by the healthcare worker HC. Although FIG. 1 illustrates a case where the second in-hospital terminal 7 is a laptop PC, the second in-hospital terminal 7 may be a desktop PC or a tablet PC. The second in-hospital terminal 7 is connected to the isolated network NW3 and communicates with the scanner 6 and a storage device 8.

The information management system 1000 includes the storage device 8.

The storage device 8 is a device that includes a database 80 and that stores the medical questionnaire answer information J1 as an electronic medical record in the database 80. The storage device 8 connects to the isolated network NW3 and communicates with the second in-hospital terminal 7.

### 1-2. Configuration of Server

Next, the configurations of the medical questionnaire management server 10 and the print server 20 will be described.

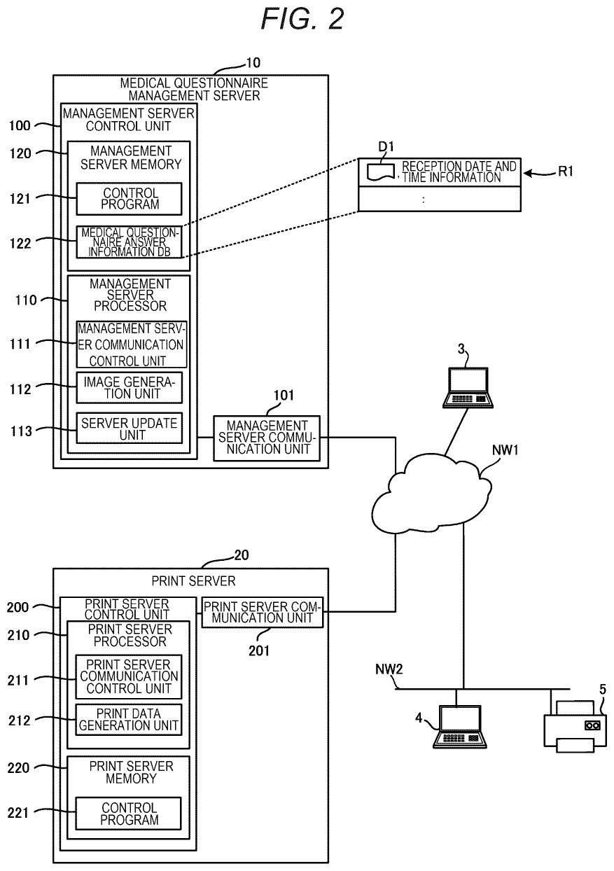

FIG. 2 shows the configurations of the medical questionnaire management server 10 and the print server 20.

First, the configuration of the medical questionnaire management server 10 will be described.

The medical questionnaire management server 10 includes a management server control unit 100 and a management server communication unit 101.

The management server communication unit 101 has communication hardware conforming to a predetermined communication standard, such as a communication circuit, and communicates with the print system 2, the patient terminal 3, and the first in-hospital terminal 4 under the control of the management server control unit 100.

The management server control unit 100 includes a management server processor 110 such as a CPU (central processing unit) or an MPU (micro-processing unit), and a management server memory 120. The management server communication unit 101 is coupled to the management server control unit 100.

The management server memory 120 is a memory that stores programs and data. The management server memory 120 stores a control program 121, a medical questionnaire answer information DB (database) 122, and data processed by the management server processor 110, or the like. The management server memory 120 has a nonvolatile storage area. The management server memory 120 also has a volatile storage area, which forms a work area of the management server processor 110. The management server memory 120 is configured with, for example, a ROM (read-only memory) or a RAM (random-access memory).

The medical questionnaire answer information DB 122 is a database having a record R1 for each medical questionnaire answer information J1. The record R1 includes the medical questionnaire answer information data D1 and consultation date and time information indicating the date and time when the medical questionnaire answer information data D1 is received from the patient terminal 3, or the like.

The management server processor 110 reads and executes the control program 121 stored in the management server memory 120 and thus functions as a management server communication control unit 111, an image generation unit 112, and a server update unit 113.

The management server communication control unit 111 communicates with the print system 2, the patient terminal 3, and the first in-hospital terminal 4 via the management server communication unit 101.

When the management server communication control unit 111 receives the medical questionnaire answer information data D1 from the patient terminal 3, the image generation unit 112 generates a print image PG to be printed by the printing device 5, based on the medical questionnaire answer information data D1 received by the management server communication control unit 111.

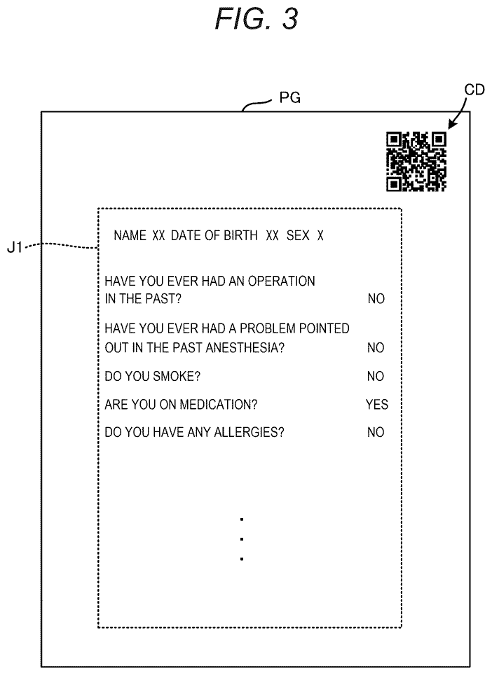

FIG. 3 shows an example of the print image PG.

The print image PG includes the medical questionnaire answer information J1.

The print image PG also includes a code CD. In FIG. 3, a QR code (registered trademark), which is a two-dimensional code, is shown as an example of the code CD, but the code CD included in the print image PG may be a one-dimensional code.

The image generation unit 112 generates a job ID (identification) when generating the print image PG. The job ID is identification information for identifying the printed object on which the medical questionnaire answer information J1 is printed. The image generation unit 112 generates a job ID according to serial numbers defining an order rule of consecutive numbers. For example, when the image generation unit 112 previously generates a job ID “001”, the image generation unit 112 generates a job ID “002” this time.

Hereinafter, the printed object on which the medical questionnaire answer information J1 is printed is referred to as a “medical questionnaire answer printed object” as appropriate.

The serial numbers are an example of a “predetermined order rule”.

Upon generating the job ID, the image generation unit 112 generates a code CD in which the generated job ID and the medical questionnaire answer information J1 indicated by the medical questionnaire answer information data D1 are recorded. Then, the image generation unit 112 generates a print image PG including the generated code CD and the medical questionnaire answer information J1 indicated by the medical questionnaire answer information data D1.

Upon generating the print image PG, the image generation unit 112 outputs the image data of the generated print image PG to the management server communication control unit 111. Upon receiving the image data of the print image PG, the management server communication control unit 111 transmits the received image data of the print image PG to the print system 2 together with information designating the printing device 5 as the print destination.

The server update unit 113 updates the medical questionnaire answer information DB 122. When the management server communication control unit 111 receives the medical questionnaire answer information data D1 from the patient terminal 3, the server update unit 113 generates a record R1 including the medical questionnaire answer information data D1, and stores the generated record R1 in the medical questionnaire answer information DB 122.

Next, the configuration of the print server 20 will be described.

The print server 20 includes a print server control unit 200 and a print server communication unit 201.

The print server control unit 200 is an example of a “server control unit”.

The print server communication unit 201 has communication hardware conforming to a predetermined communication standard, such as a communication circuit, and communicates with the medical questionnaire management system 1 and the printing device 5 under the control of the print server control unit 200.

The print server control unit 200 includes a print server processor 210 such as a CPU (central processing unit) or an MPU (micro-processing unit), and a print server memory 220. The print server communication unit 201 is coupled to the print server control unit 200.

The print server memory 220 is a memory that stores programs and data. The print server memory 220 stores a control program 221 and data processed by the print server processor 210, or the like. The print server memory 220 has a nonvolatile storage area. The print server memory 220 also has a volatile storage area, which forms a work area of the print server processor 210. The print server memory 220 is configured with, for example, a ROM (read-only memory) or a RAM (random-access memory).

The print server processor 210 reads and executes the control program 221 stored in the print server memory 220 and thus functions as a print server communication control unit 211 and a print data generation unit 212.

The print server communication control unit 211 communicates with the medical questionnaire management system 1 and the printing device 5 via the print server communication unit 201.

The print data generation unit 212 generates print data D2, based on the image data received from the medical questionnaire management system 1. Upon generating the print data D2, the print data generation unit 212 outputs the generated print data D2 to the print server communication control unit 211. Upon receiving the print data D2, the print server communication control unit 211 transmits the received print data D2 to the printing device 5 designated by the medical questionnaire management system 1.

### 1-3. Configuration of Second In-Hospital Terminal

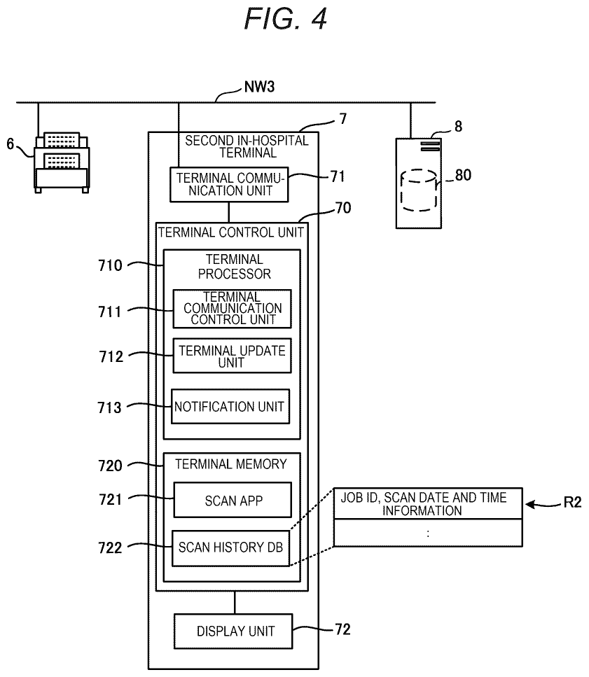

Next, the configuration of the second in-hospital terminal 7 will be described. FIG. 4 shows the configuration of the second in-hospital terminal 7.

The second in-hospital terminal 7 includes a terminal control unit 70, a terminal communication unit 71, and a display unit 72.

The terminal control unit 70 is an example of a “management device control unit”.

The terminal communication unit 71 includes communication hardware conforming to a predetermined communication standard, such as a communication circuit, and communicates with the scanner 6 and the storage device 8 under the control of the terminal control unit 70.

The display unit 72 includes a display, and displays information on the display under the control of the terminal control unit 70.

The terminal control unit 70 includes a terminal processor 710 such as a CPU (central processing unit) or an MPU (micro-processing unit), and a terminal memory 720. The terminal communication unit 71 and the display unit 72 are coupled to the terminal control unit 70.

The terminal processor 710 is an example of a “processor”.

The terminal memory 720 is a memory that stores programs and data. The terminal memory 720 stores a scan application program (hereinafter referred to as “scan APP”) 721, a scan history DB 722, data processed by the terminal processor 710, and the like. The terminal memory 720 has a nonvolatile storage area. The terminal memory 720 also has a volatile storage area, which forms a work area of the terminal processor 710. The terminal memory 720 includes, for example, a ROM (read-only memory) or a RAM (random-access memory).

The scan APP 721 is an example of a “program”.

The scan APP 721 is an application program for controlling scanning by the scanner 6, and performs processing of generating scan data D3 of an image read by the scanner 6, processing of saving the scan data in a desired saving location, and the like.

The scan history DB 722 is a database that manages a scan history. The scan history DB 722 includes a record R2 for each job ID. The record R2 includes a job ID and scan date and time information indicating the date and time of scan. In the scan history DB 722, records R2 are stored in the order of job IDs.

The terminal processor 710 reads and executes the scan APP 721 stored in the terminal memory 720 and thus functions as a terminal communication control unit 711, a terminal update unit 712, and a notification unit 713.

The terminal communication control unit 711 communicates with the scanner 6 and the storage device 8 via the terminal communication unit 71.

The terminal update unit 712 updates the scan history DB 722. When the terminal communication unit 71 receives the scan data D3 of the medical questionnaire answer printed object, the terminal update unit 712 acquires the job ID from the scan image represented by the scan data D3 of the medical questionnaire answer printed object. The scan image represented by the scan data D3 of the medical questionnaire answer printed object includes the print image PG. Therefore, the terminal update unit 712 reads the job ID from the code CD of the print image PG included in the scan image and thus acquires the job ID. Upon acquiring the job ID from the scan data D3 of the medical questionnaire answer printed object, the terminal update unit 712 generates a record R2 including the acquired job ID and the scan date and time information and stores the generated record R2 in the scan history DB 722. The date and time of scan indicated by the scan date and time information included in the record R2 may be the date and time when the second in-hospital terminal 7 receives the scan data D3, the date and time when the record R2 is generated, or the date and time when the information is acquired from the code CD.

The notification unit 713 displays information on the display unit 72 and thus notifies the healthcare worker HC of the information.

### 1-4. Operation of Information Management System

Next, the operation of each unit of the information management system 1000 will be described.

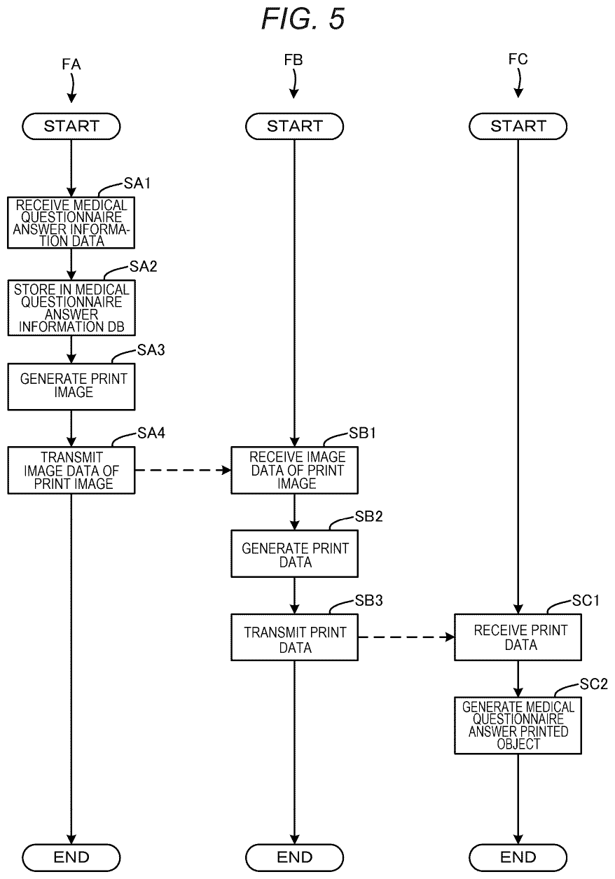

FIG. 5 is a flowchart showing the operation of each unit of the information management system 1000. In FIG. 5, a flowchart FA shows the operation of the medical questionnaire management system 1, a flowchart FB shows the operation of the print system 2, and a flowchart FC shows the operation of the printing device 5.

When the patient terminal 3 transmits the medical questionnaire answer information data D1 to the medical questionnaire management system 1, the management server communication control unit 111 receives the medical questionnaire answer information data D1 from the patient terminal 3 (step SA1).

Next, the server update unit 113 stores the record R1 including the medical questionnaire answer information data D1 received in step SA1, in the medical questionnaire answer information DB 122 (step SA2).

Next, the image generation unit 112 generates the print image PG, based on the medical questionnaire answer information data D1 received in step SA1 (step SA3).

Next, the management server communication control unit 111 transmits the image data of the print image PG generated in step SA3 to the print system 2 (step SA4).

As shown in flowchart FB, the print server communication control unit 211 receives the image data of the print image PG from the medical questionnaire management system 1 (step SB1).

Next, the print data generation unit 212 generates the print data D2 for printing the print image PG represented by the image data received in step SB1, based on the image data received in step SB1 (step SB2).

Next, the print server communication control unit 211 transmits the print data D2 generated in step SB2 to the printing device 5 (step SB3).

As shown in flowchart FC, upon receiving the print data D2 from the print system 2 (step SC1), the printing device 5 executes printing based on the received print data D2, and generates a medical questionnaire answer print product on which the print image PG generated in step SA3 is printed (step SC2).

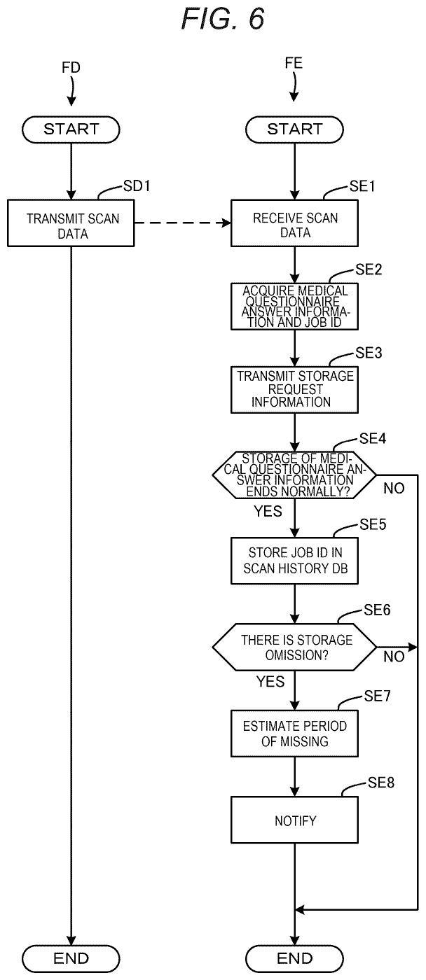

When the medical questionnaire answer printed object is generated, the healthcare worker HC sets the medical questionnaire answer printed object on the scanner 6, and performs an operation to the scanner 6 or the scan application so as to cause the scanner 6 to scan the medical questionnaire answer printed object. Next, referring to FIG. 6, the operation of the information management system 1000 when the scanner 6 scans the medical questionnaire answer printed object will be described.

FIG. 6 is a flowchart showing the operation of each unit of the information management system 1000. In FIG. 6, a flowchart FD shows the operation of the scanner 6, and a flowchart FE shows the operation of the second in-hospital terminal 7.

Upon scanning, the scanner 6 transmits the scan data D3 of the medical questionnaire answer printed object to the second in-hospital terminal 7 (step SD1).

As shown in flowchart FE, the terminal communication control unit 711 receives the scan data D3 of the medical questionnaire answer printed object (step SE1).

Next, the terminal communication control unit 711 acquires the medical questionnaire answer information J1 and the job ID from the code CD contained in the scan image represented by the scan data D3 received in step SE1 (step SE2).

Next, the terminal communication control unit 711 transmits storage request information to the storage device 8 (step SE3). The storage request information is information requesting the database 80 to store the medical questionnaire answer information J1 acquired in step SE2. The storage request information includes medical questionnaire answer information data D1 representing the medical questionnaire answer information J1 acquired in step SE2.

Upon receiving the storage request information, the storage device 8 stores the medical questionnaire answer information data D1 in the database 80 and thus stores the medical questionnaire answer information J1 in the database 80. When the storage of the medical questionnaire answer information J1 is normally finished, the storage device 8 sends back a response to the storage request information, indicating that the storage of the medical questionnaire answer information J1 is normally finished.

The terminal update unit 712 determines whether the storage of the medical questionnaire answer information J1 is finished normally (step SE4). When a response indicating that the storage is finished normally is received from the storage device 8, the terminal update unit 712 makes a positive determination in step SE4, and when a response indicating that the storage is not finished normally is received from the storage device 8, the terminal update unit 712 makes a negative determination in step SE4.

When the terminal update unit 712 determines that storage of the medical questionnaire answer information J1 is not finished normally (NO in step SE4), the terminal update unit 712 ends the processing. When the terminal update unit 712 makes a negative determination in step SA4, the notification unit 713 may give a notification that the storage of the medical questionnaire answer information J1 is not finished normally.

Meanwhile, when the terminal update unit 712 determines that the storage of the medical questionnaire answer information J1 is finished normally (YES in step SE4), the terminal update unit 712 acquires the job ID from the scan data D3 received in step SE1, and stores the record R2 including the acquired job ID in the scan history DB 722 (step SE5).

Next, the notification unit 713 determines whether there is a storage omission of the medical questionnaire answer information J1 in the database 80 (step SE6).

Step SE6 will be described in detail.

The notification unit 713 determines whether a job ID having a number consecutive to the job ID of the record R2 stored in step SE5 is stored in the scan history DB 722. For example, when the job ID of the record R2 stored in step SE5 is “005” and there is no job ID “004” among the job IDs stored in the scan history DB 722, the notification unit 713 determines that a job ID having a number consecutive to the job ID of the record R2 stored in step SE5 is not stored in the scan history DB 722.

When the notification unit 713 determines that a job ID having a number consecutive to the job ID of the record R2 stored in step SE5 is stored in the scan history DB 722, the notification unit 713 makes a positive determination in step SE6. Meanwhile, when the notification unit 713 determines that a job ID having a number consecutive to the job ID of the record R2 stored in step SE5 is not stored in the scan history DB 722, the notification unit 713 makes a negative determination in step SE6.

When the notification unit 713 determines that there is no storage omission of the medical questionnaire answer information J1 in the database 80 (NO in step SE6), the terminal processor 710 ends this processing.

Meanwhile, when it is determined that there is a storage omission of the medical questionnaire answer information J1 in the database 80 (YES in step SE6), the notification unit 713 estimates the period of missing of the medical questionnaire answer printed object on which the medical questionnaire answer information J1 that is omitted from the storage in the database 80 is printed (step SE7).

Step SE7 will be described in detail.

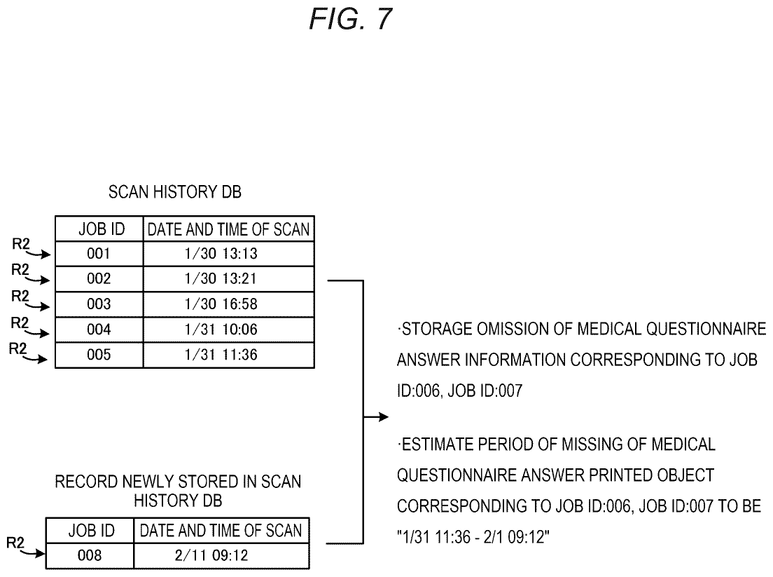

FIG. 7 illustrates the estimation of the period of missing.

The notification unit 713 estimates the period of missing for each job ID corresponding to the medical questionnaire answer information J1 omitted from the storage in the database 80.

FIG. 7 illustrates a case where the scan history DB 722 includes a record R2 of a job ID “001”, a record R2 of a job ID “002”, a record R2 of a job ID “003”, a record R2 of a job ID “004”, and a record R2 of a job ID “005”.

FIG. 7 also illustrates a case where a record R2 of a job ID “008” is newly stored in the scan history DB 722.

In the example shown in FIG. 7, when the record R2 of the job ID “008” is stored, a record R2 of a job ID “006” and a record R2 of a job ID “007” are not stored in the scan history DB 722. Therefore, in the case of the example shown in FIG. 7, the notification unit 713 specifies the job ID “006” and the job ID “007” as the job ID corresponding to the medical questionnaire answer information J1 omitted from the storage in the database 80. In the case of the example shown in FIG. 7, the notification unit 713 estimates the period of missing for the specified job ID “006” and job ID “007”.

In the case of the example shown in FIG. 7, the notification unit 713 estimates the period of missing of the medical questionnaire answer printed object corresponding to the job ID “006” and the period of missing of the medical questionnaire answer printed object corresponding to the job ID “007” in the following manner. In the example shown in FIG. 7, the record R2 stored in the scan history DB 722 before the record R2 of the job ID “008” is the record R2 of the job ID “005”. Therefore, in the case of the example shown in FIG. 7, the notification unit 713 estimates the period of missing of the job ID “006” and the job ID “007” to be the period from the date and time of scan indicated by the scan date and time information included in the record R2 of the job ID “005” to the date and time of scan indicated by the scan date and time information included in the record R2 of the job ID “008”. That is, in the case of the example shown in FIG. 7, the notification unit 713 estimates the period of missing of the job ID “006” and the job ID “007” to be “1/31 11:36-2/1 09:12”.

Back to the description with reference to FIG. 6, upon estimating the period of missing, the notification unit 713 gives a notification of the storage omission in the database 80 (step SE8).

In step SE8, the notification unit 713 causes the display unit 72 to display a notification screen TG and thus gives a notification of the storage omission in the database 80.

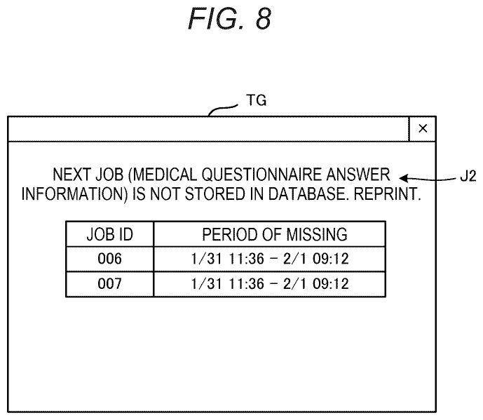

FIG. 8 shows an example of the notification screen TG.

The notification screen TG is a screen that displays a list of combinations of a job ID corresponding to the medical questionnaire answer information J1 omitted from the storage in the database 80 and the period of missing of the job ID.

The notification screen TG is a screen that displays reprint information J2 that prompts the user to reprint the medical questionnaire answer information J1 omitted from the storage in the database 80.

The notification unit 713 generates a notification screen TG displaying a combination of a job ID and a period of missing for each job ID specified when estimating the period of missing, and causes the display unit 72 to display the generated notification screen TG.

### 1-5. Configuration and Effects

As described above, the information management system 1000 includes the medical questionnaire management system 1 that manages the medical questionnaire answer information J1. The information management system 1000 also includes the print system 2 that causes the printing device 5 to print the medical questionnaire answer information J1 acquired from the medical questionnaire management system 1. The information management system 1000 includes the second in-hospital terminal 7 that acquires the medical questionnaire answer information J1 from the scan data D3 of the medical questionnaire response printed object with the medical questionnaire answer information J1 printed thereon, and that stores the acquired medical questionnaire answer information J1 in the database 80 on the isolated network NW3 isolated from the external network NW1. The print system 2 causes the printing device 5 to generate a medical questionnaire answer printed object with the job ID of a serial number printed thereon. The second in-hospital terminal 7 acquires the job ID from the scan data D3 of the medical questionnaire answer printed object, and gives a notification of the storage omission of the medical questionnaire answer information J1 in the database 80, based on the acquired job ID.

Thus, when there is a storage omission of the medical questionnaire answer information J1 in the database 80 due to a loss of the medical questionnaire answer printed object, a multi-feed occurring in the scanner 6, or the like, the healthcare worker HC can grasp that there is a storage omission of the medical questionnaire answer information J1 in the database 80. Therefore, the healthcare worker HC gives an instruction to reprint from the in-hospital terminal 4, causes the reprinted printed object to be scanned, thus stores the medical questionnaire answer information J1 in the database 80, and therefore can cope with the storage omission of the medical questionnaire answer information J1 in the database 80.

When there is a job ID that is not a serial number among the acquired job IDs, the second in-hospital terminal 7 determines that there is a storage omission of the medical questionnaire answer information J1 in the database 80, and gives a notification of the storage omission of the medical questionnaire answer information J1 in the database 80.

Thus, by utilizing the fact that the job ID is a serial number, it can be accurately determined that there is a storage omission of the medical questionnaire answer information J1 in the database 80. Therefore, the omission of storage of the medical questionnaire answer information J1 in the database 80 can be notified of in an appropriate situation. The appropriate situation refers to a situation where there is a storage omission of the medical questionnaire answer information J1 in the database 80.

The printing device 5 generates a medical questionnaire answer printed object with the medical questionnaire answer information J1 printed thereon in a code format. The second in-hospital terminal 7 acquires the medical questionnaire answer information J1 from the code CD included in the scan image represented by the scan data D3 of the medical questionnaire answer printed object.

Thus, the medical questionnaire answer information J1 can be acquired without performing character recognition on the printing surface of the medical questionnaire answer printed object, and therefore the processing load when storing the medical questionnaire answer information J1 in the database 80 can be reduced.

The second in-hospital terminal 7 estimates the period of missing of the medical questionnaire answer printed object on which the medical questionnaire answer information J1 omitted from the storage in the database 80 is printed, based on the time when the medical questionnaire answer printed object is scanned, and gives a notification of the estimated period of missing.

Thus, the healthcare worker HC can easily grasp during which period the medical questionnaire answer printed object on which the medical questionnaire answer information J1 omitted from the storage in the database 80 is printed is provided. Therefore, the healthcare worker HC can accurately cope with the storage omission of the medical questionnaire answer information J1 in the database 80, and the likelihood that the storage omission of the medical questionnaire answer information J1 in the database 80 can be accurately solved can be increased.

The second in-hospital terminal 7 includes the terminal control unit 70 that acquires the job ID from the scan data D3 of the medical questionnaire answer printed object with the job ID of a serial number printed thereon, and that gives a notification of the storage omission of the medical questionnaire answer information J1 in the database 80 on the isolated network NW3, based on the acquired job ID.

Thus, effects similar to those of the information management system 1000 described above are achieved.

The scan APP 721 causes the terminal processor 710 of the second in-hospital terminal 7 to acquire the job ID from the scan data D3 of the medical questionnaire answer printed object with the job ID of a serial number printed thereon, and to gives a notification of the storage omission of the medical questionnaire answer information J1 in the database 80 on the isolated network NW3, based on the acquired job ID.

Thus, effects similar to those of the information management system 1000 described above are achieved.

The print server 20 includes the print server control unit 200. The print server control unit 200 causes the printing device 5 to print the medical questionnaire answer printed object on which the job ID acquired when the second in-hospital terminal 7 connected to the isolated network NW3 gives a notification of the storage omission of the medical questionnaire answer information J1 in the database 80 on the isolated network NW3 is printed. The job ID printed on the medical questionnaire answer printed object is serial number information.

Thus, effects similar to those of the information management system 1000 described above are achieved.

### 2. Second Embodiment

### 2-1. Configuration of Server

A second embodiment will now be described.

As compared with the first embodiment, the second embodiment is different from the first embodiment in the configuration of the medical questionnaire management server 10 and the print server 20.

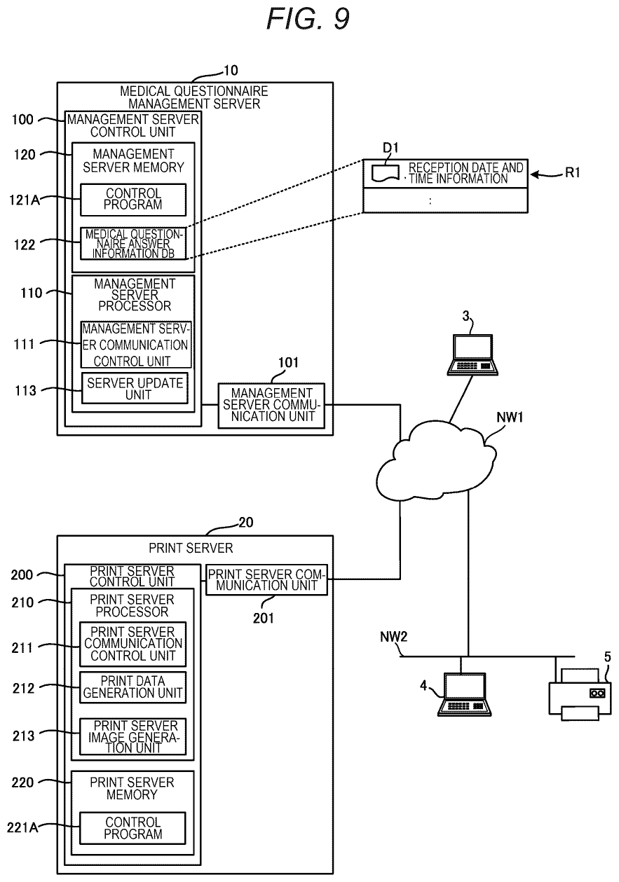

FIG. 9 shows the configuration of the medical questionnaire management server 10 and the print server 20 in the second embodiment.

In the description of the configuration of the medical questionnaire management server 10 and the print server 20 in the second embodiment, differences from the first embodiment will be described.

In the second embodiment, the management server memory 120 stores a control program 121A instead of the control program 121. The control program 121A is a program that causes the management server processor 110 to function as the management server communication control unit 111 and the server update unit 113. That is, in the second embodiment, the management server processor 110 does not function as the image generation unit 112.

In the second embodiment, the print server memory 220 stores a control program 221A instead of the control program 221. The control program 221A is a program that causes the print server processor 210 to function as the print server communication control unit 211, the print data generation unit 212, and a print server image generation unit 213. That is, in the second embodiment, the print server processor 210 further functions as the print server image generation unit 213.

In the second embodiment, the print server communication control unit 211 receives the medical questionnaire answer information data D1 from the medical questionnaire management system 1. Similarly to the image generation unit 112, the print server image generation unit 213 generates the print image PG, based on the medical questionnaire answer information data D1 received by the print server communication control unit 211. Upon generating the print image PG, the print server image generation unit 213 outputs the image data of the generated print image PG to the print data generation unit 212. Upon receiving the image data of the print image PG, the print data generation unit 212 generates print data for printing the print image PG, based on the received image data of the print image PG. The print data D2 generated by the print data generation unit 212 is transmitted to the printing device 5.

### 2-2. Operation of Information Management System

Next, the operation of each unit of the information management system 1000 according to the present embodiment will be described.

As compared with the first embodiment, the second embodiment is different from the first embodiment in the operations of the medical questionnaire management server 10 and the print server 20 until the printing device 5 generates the medical questionnaire answer printed object.

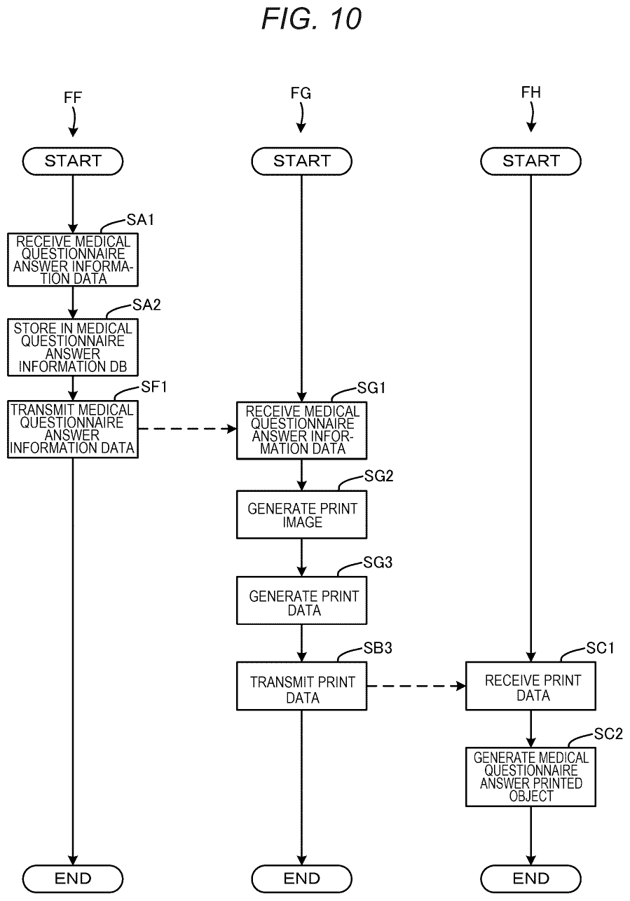

FIG. 10 is a flowchart showing the operation of each unit of the information management system 1000. In FIG. 10, a flowchart FF shows the operation of the medical questionnaire management system 1, a flowchart FG shows the operation of the print system 2, and a flowchart FH shows the operation of the printing device 5.

In each flowchart shown in FIG. 10, the same steps as those in each flowchart shown in FIG. 5 are denoted by the same step numbers, and the detailed description thereof is omitted.

As shown in flowchart FF, the management server communication control unit 111 transmits the medical questionnaire answer information data D1 received in step SA1 to the print system 2 (step SF1).

As shown in flowchart FG, the print server communication control unit 211 receives the medical questionnaire answer information data D1 from the medical questionnaire management system 1 (step SG1).

Next, the print server image generation unit 213 generates the print image PG, based on the medical questionnaire answer information data D1 received in step SG1 (step SG2).

Next, the print data generation unit 212 generates the print data D2 for printing the print image PG generated in step SG2 (step SG3).

### 2-3. Effects

According to the present embodiment, effects similar to those of the first embodiment are achieved.

### 3. Other Embodiments

The embodiments described above represent one aspect and any modifications and applications thereof can be made.

In the above-described embodiments, the serial number is described as an example of the “predetermined order rule”. That is, in the above-described embodiments, the job ID is generated as a serial number. In another embodiment, the “predetermined order rule” is not limited to the serial number, and may be another order rule such as an alphabetical character order.

When a plurality of organizations are established in the hospital H and the printing device 5 is installed for each organization, in another embodiment, the job ID may be an ID distinguished for each organization. In this case, the job ID includes organization information that indicates an organization and is different for each organization. For example, the job ID corresponding to the organization of the anesthesiology department is “ANE001” and includes organization information of “ANE”. In this another embodiment, the medical questionnaire answer information J1 is divided for each organization. Then, the print system 2 causes the printing device 5 installed in the corresponding organization to print the divided medical questionnaire answer information J1. When causing the printing device 5 to print, the print system 2 generates the job ID including the organization information of the corresponding organization, generates the print image PG including the code CD with the generated job ID recorded therein, and causes the printing device 5 to print the print image PG. According to this another embodiment, since the organization information is included in the job ID, a notification of the storage omission of the medical questionnaire answer information J1 in the database 80 can be given for each organization.

In the above-described embodiments, a configuration in which the record R2 including the job ID corresponding to the medical questionnaire answer information J1 stored in the database 80 is stored in the scan history DB 722 is employed. In another embodiment, a configuration in which the record R2 including the job ID acquired from the scan data D3 is stored in the scan history DB 722, regardless of the completion of storage in the database 80, may be employed.

In the above-described embodiments, a configuration in which a notification of the storage omission in the database 80 is given by a display is employed, but the form of the notification of the storage omission in the database 80 is not limited to the display and may be other forms such as a voice output or transmission of an e-mail.

In the above-described embodiments, a case where the number of scanners 6 connected to the isolated network NW3 and the number of printing devices 5 connected to the external connection network NW2 are one is described, but the number of scanners 6 connected to the isolated network NW3 and the number of printing devices 5 connected to the external connection network NW2 are not limited to one and may be plural.

In the above-described embodiments, a case where the information management system 1000 is applied to the hospital H is described as an example. However, the target to which the information management system 1000 is applied is not limited to the hospital H, and the information management system 1000 may also be applied to a facility or a subject that manages personal information, confidential information, and the like by an isolated network. In this case, the “management system” manages “management information” such as personal information and confidential information, and the “management device” is connected to the isolated network and stores the “management information” such as personal information and confidential information in a “database” on the isolated network. For example, the targets other than the hospital H to which the information management system 1000 is applied are government and public agencies, police, and the like.

In the above-described embodiments, a configuration in which the job ID and the medical questionnaire answer information J1 are acquired from the code CD by printing the code CD is employed. In another embodiment, a configuration in which the job ID and the medical questionnaire answer information J1 are printed instead of printing the code CD and in which the job ID and the medical questionnaire answer information J1 are acquired from a scan image by OCR (optical character recognition, may be employed.

In another embodiment, the functions of the management server processor 110, the print server processor 210, and the terminal processor 710 may be implemented by a plurality of processors or a semiconductor chip.

The respective units of the medical questionnaire management server 10, the print server 20, and the second in-hospital terminal 7 shown in FIGS. 2, 4, and 9 are examples, and are not particularly limited. Pieces of hardware individually corresponding to the units need not necessarily be installed, and as a matter of course, one processor can be configured to execute a program to implement the functions of the units. A part of the functions implemented by software may be implemented by hardware or a part of the functions implemented by hardware may be implemented by software. In addition, the specific detailed configuration of each of the medical questionnaire management server 10, the print server 20, and the second in-hospital terminal 7 can be freely changed.

The steps of the operations illustrated in FIGS. 5, 6, and 10 are provided by dividing the operations according to the main processing content in order to facilitate the understanding of the operation of each unit of the information management system 1000, and are not limited by the way the operations are divided into processing elements and the names thereof. The operations may be divided into more steps according to the processing content. The operations may also be divided so that one step includes more processing. The order of the steps may be appropriately changed.

### 4. Summary of Present Disclosure

The present disclosure will be summarized below in the form of appendices.

### Appendix 1

An information management system includes: a management system that manages management information; a print system that causes a printing device to print the management information acquired from the management system; and a management device that acquires the management information from scan data of a printed object with the management information printed thereon, and stores the acquired management information in a database on an isolated network isolated from an external network, the print system causing the printing device to generate the printed object with identification information in accordance with a predetermined order rule printed thereon, the management device acquiring the identification information from the scan data of the printed object and giving a notification of a storage omission of the management information in the database, based on the acquired identification information.

This configuration enables the user of the management device to grasp that there is a storage omission of management information in the database on the isolated network when there is a storage omission of the management information in the database on the isolated network due to a loss of printed object, a multi-feed occurring in the scanner, or the like. Thus, the user of the management device causes a reprinted printed object to be scanned, thus stores the management information in the database, and therefore can cope with the storage omission of the management information in the database.

### Appendix 2

In the information management system according to Appendix 1, when the acquired identification information includes the identification information that is not in accordance with the predetermined order rule, the management device determines that there is a storage omission of the management information in the database, and gives a notification of the storage omission of the management information in the database.

With this configuration, by utilizing the fact that the identification information is in accordance with the predetermined order rule, it can be accurately determined that there is a storage omission of the management information in the database. Therefore, the storage omission of the management information in the database can be notified of in an appropriate situation.

### Appendix 3

In the information management system according to Appendix 1 or 2, the printing device generates the printed object with the management information printed thereon in a code format, and the management device acquires the management information from a code included in a scan image represented by scan data of the printed object.

With this configuration, since the management information can be acquired without performing character recognition on the printing surface of the printed object, the processing load when storing the management information in the database can be reduced.

### Appendix 4

In the information management system according to one of Appendices 1 to 3, the management device estimates a period of missing of the printed object on which the management information omitted from the storage in the database is printed, based on a time when the printed object is scanned, and gives a notification of the estimated period of missing.

This configuration enables the user of the management device to easily grasp during which period the printed object on which the management information omitted from the storage in the database is printed is provided. Thus, the user of the management device can accurately cope with the storage omission of the management information in the database, and the likelihood that the storage omission of the management information in the database can be accurately solved can be increased.

### Appendix 5

In the information management system according to one of Appendices 1 to 4, the identification information includes organization information indicating an organization established in a facility where the isolated network is built.

With this embodiment, since the organization information is included in the identification information, a notification of the storage omission of the management information in the database can be given for each organization.

### Appendix 6

A management device is provided, the management device being connected to an isolated network isolated from an external network to which a management system that manages management information and a print system that causes a printing device to print the management information acquired from the management system are connected, the management device including a management device control unit that acquires identification information in accordance with a predetermined order rule from scan data of a printed object with the identification information printed thereon, and gives a notification of a storage omission of the management information in a database on the isolated network, based on the acquired identification information.

This configuration achieves effects similar to those of the information management system according to the Appendix 1.

### Appendix 7

A non-transitory computer-readable storage medium storing a program is provided, the program causing a processor of a management device connected to an isolated network isolated from an external network to which a management system that manages management information and a print system that causes a printing device to print the management information acquired from the management system are connected, to acquire identification information in accordance with a predetermined order rule from scan data of a printed object with the identification information printed thereon, and give a notification of a storage omission of the management information in a database on the isolated network, based on the acquired identification information.

This configuration achieves effects similar to those of the information management system according to the Appendix 1.

### Appendix 8

A server is provided, the server being connected to a management system that manages management information, via an external network, and causing a printing device to print the management information acquired from the management system, the server including a server control unit that causes the printing device to print a printed object on which identification information acquired when the management device connected to an isolated network isolated from the external network gives a notification of a storage omission of the management information in a database on the isolated network is printed, the identification information printed on the printed object being information in accordance with a predetermined order rule.

This configuration achieves effects similar to those of the information management system according to the Appendix 1.

What is claimed is:

1. An information management system comprising:
a management system that manages management information;
a print system that causes a printing device to print the management information acquired from the management system; and
a management device that acquires the management information from scan data of a printed object with the management information printed thereon, and stores the acquired management information in a database on an isolated network isolated from an external network,
the print system causing the printing device to generate the printed object with identification information in accordance with a predetermined order rule printed thereon,
the management device acquiring the identification information from the scan data of the printed object and giving a notification of a storage omission of the management information in the database, based on the acquired identification information.

2. The information management system according to claim 1, wherein
when the acquired identification information includes the identification information that is not in accordance with the predetermined order rule, the management device determines that there is a storage omission of the management information in the database, and gives a notification of the storage omission of the management information in the database.

3. The information management system according to claim 1, wherein
the printing device generates the printed object with the management information printed thereon in a code format, and
the management device acquires the management information from a code included in a scan image represented by scan data of the printed object.

4. The information management system according to claim 1, wherein
the management device estimates a period of missing of the printed object on which the management information omitted from the storage in the database is printed, based on a time when the printed object is scanned, and gives a notification of the estimated period of missing.

5. The information management system according to claim 1, wherein
the identification information includes organization information indicating an organization established in a facility where the isolated network is built.

6. A management device connected to an isolated network isolated from an external network to which a management system that manages management information and a print system that causes a printing device to print the management information acquired from the management system are connected,
the management device comprising:
a management device control unit that acquires identification information in accordance with a predetermined order rule from scan data of a printed object with the identification information printed thereon, and gives a notification of a storage omission of the management information in a database on the isolated network, based on the acquired identification information.

7. A non-transitory computer-readable storage medium storing a program, the program causing a processor of a management device connected to an isolated network isolated from an external network to which a management system that manages management information and a print system that causes a printing device to print the management information acquired from the management system are connected, to:
acquire identification information in accordance with a predetermined order rule from scan data of a printed object with the identification information printed thereon, and
give a notification of a storage omission of the management information in a database on the isolated network, based on the acquired identification information.
